(19) United States
(12) Patent Application Publication
(10) Pub. No.: US 2016/0166821 A1
(43) Pub. Date: Jun. 16, 2016

(54) DEVICES FOR RESTRAINING MOVEMENT OF ELONGATED MEDICAL IMPLEMENTS AND RELATED SYSTEMS AND METHODS

(71) Applicant: Intermountain Invention Management, LLC, Salt Lake City, UT (US)

(72) Inventors: J. Peter Weiss, Salt Lake City, UT (US); Troy J. Orr, Draper, UT (US)

(21) Appl. No.: 14/970,312

(22) Filed: Dec. 15, 2015

Related U.S. Application Data
(60) Provisional application No. 62/092,242, filed on Dec. 15, 2014.

(51) Int. Cl. A61M 39/06; A61M 25/00; A61M 25/01

(52) U.S. Cl. CPC A61M 39/0613; A61M 25/01; A61M 25/0097; A61M 2039/062

(57) ABSTRACT

Devices are disclosed that can be used with a medical instrument that comprises an elongated sheath and a hub that comprises a sideport. The hub and the sheath of the medical instrument are configured to permit at least a portion of an elongated medical implement to pass through them. The devices can include a plurality of engagement members that cooperate to define an opening through which the elongated medical implement can pass and a biasing surface. The devices can further include an adapter that includes an attachment mechanism to directly engage the sideport to secure the adapter to the medical instrument and a locking member for coupling with the adapter. The adapter and the locking member can interact to advance the plurality of engagement members along the biasing surface to transition the engagement members from an open state to a locked state.

### CROSS-REFERENCE TO RELATED APPLICATIONS

This application claims the benefit under 35 U.S.C. §119(e) of U.S. Provisional Patent Application No. 62/092,242, titled DEVICES FOR RESTRAINING MOVEMENT OF ELONGATED MEDICAL INSTRUMENTS AND RELATED SYSTEMS AND METHODS, filed on Dec. 15, 2014, the entire contents of which are hereby incorporated by reference herein.

### BACKGROUND

The present disclosure relates to devices for constraining movement of elongated medical implements and related systems and methods. The devices, systems, and methods provide advancements over some known systems that are used with certain elongated medical implements, and in some instances, provide restraint for elongated medical implements in situations where such was previously absent.

### BRIEF DESCRIPTION OF THE DRAWINGS

The written disclosure herein describes illustrative embodiments that are non-limiting and non-exhaustive. Reference is made to certain of such illustrative embodiments that are depicted in the figures, in which:

FIG. 1 is an exploded perspective view of an embodiment of a medical system that includes an embodiment of a medical instrument that includes a hub and also includes a sheath configured for insertion into a patient, an embodiment of an elongated medical implement, and an embodiment of a retaining device configured to restrain movement of the elongated medical implement relative to the medical instrument;

FIG. 2A is a perspective view of an embodiment of a first portion of the retaining device of FIG. 1 that may also be referred to as an adapter;

FIG. 2B is a cross-sectional view of the adapter of FIG. 2A taken along the view line 2B-2B in FIG. 2A;

FIG. 3A is a perspective view of an embodiment of a second portion of the retaining device of FIG. 1 that may also be referred to as a locking member;

FIG. 3B is a cross-sectional view of the locking member of FIG. 3A taken along the view line 3B-3B in FIG. 3A;

FIG. 4A is a perspective view of the retaining device of FIG. 1 in an open state;

FIG. 4B is a perspective view of the retaining device of FIG. 1 in a locking state;

FIG. 4C is a cross-sectional view of the retaining device in the locking state depicted in FIG. 4B taken along the view line 4C-4C in FIG. 4B;

FIG. 5A is a perspective view of a distal portion of the medical instrument and the retaining device of FIG. 1, wherein the retaining device is depicted just prior to being coupled with the medical instrument;

FIG. 5B is a perspective view of the distal portion of the medical instrument and the retaining device of FIG. 1, wherein the retaining device is depicted after having been coupled with the medical instrument;

FIG. 6 is another perspective view of the medical system of FIG. 1, wherein the retaining device is shown coupled with the medical instrument, and wherein the elongated medical implement is shown extending through the retaining device and the medical instrument;

FIG. 7 is another perspective view of the medical system of FIG. 1 with the retaining device shown in cross-section, wherein the elongated medical implement is depicted as having been advanced distally relative to the medical instrument, as compared with the position depicted in FIG. 6;

FIG. 8 is a cross-sectional view similar to that of FIG. 4C that depicts another embodiment of a retaining device;

FIG. 9 is an elevation view of another embodiment of an adapter that includes a connection arrangement that differs from that of the adapter of FIG. 1;

FIG. 10 is an elevation view of yet another embodiment of an adapter that includes a connection arrangement that differs from that of the adapter of FIG. 1;

FIG. 11A is an elevation view of still another embodiment of an adapter that includes a connection arrangement that differs from that of the adapter of FIG. 1; and

FIG. 11B is a cross-sectional view of the adapter of FIG. 11A taken along the view line 11B-11B in FIG. 11A.

### DETAILED DESCRIPTION

Many medical procedures involve the use of a medical instrument that comprises a hub and an elongated sheath. The hub often includes a valve (e.g., a hemostasis valve) therein, and the elongated sheath is inserted into the patient for access to a variety of regions within the patient. For example, in some embodiments, the sheath is inserted into a blood vessel of a patient for vascular access. Such sheaths are often referred to as introducer sheaths, and may be used in a wide variety of medical procedures. In other embodiments, the sheath is inserted into the patient to provide access to an interior of the heart, such as for introduction of an ablation catheter into the heart and for controlling the ablation catheter. In other instances, the sheath is inserted into the pericardium to provide access (e.g., sub-xiphoid access) to, for example, the epicardial surface of the heart. Such sheaths may also be referred to as introducers and are likewise used in a variety of medical procedures. For example, in some instances, the sheaths are used in electrophysiology procedures. The present disclosure pertains to the foregoing medical devices and procedures as well as other suitable medical devices and procedures.

In many of the foregoing procedures, an elongated medical implement is introduced into the patient via the medical device. In particular, the medical implement may be introduced into the patient by being inserted through the hub and through a lumen defined by the sheath. The elongated medical implement can be, for example, a catheter or other suitable device. A wide variety of catheters are used in numerous types of procedures. In many of such procedures, it may be desirable to restrain movement of the elongated medical implement. For example, it may be desirable to limit movement of a distal portion of the elongated medical implement relative to a distal end of the sheath portion of the medical device. Such limitation of movement can include fixing a maximum distance beyond a distal end of the sheath to which the elongated medical implement extends. Alternatively or additionally, the movement can include fixing the elongated medical implement relative to the medical device (e.g., relative to the sheath) such that the elongated medical implement does not rotate about a longitudinal axis of the combined medical device/elongated medical implement system.

For example, in certain embodiments, a cardiac ablation procedure may call for the use of a steerable sheath medical device. Such a device can provide a passageway through which an ablation catheter can be introduced into a desired region. For example, steerable sheaths may be used for endovascular or endocardial access. The sheaths may be used, for example, to assist in moving a catheter within and/or around an organ, such as, for example, the heart. The steerable sheath can, in some arrangements, provide bi-directional steering by rotating a distal portion of the distal end of the catheter, which thereby rotates a distal portion of the ablation catheter to reach sites that would otherwise be difficult to reach, and which might otherwise require changing out fixed-configuration sheaths to achieve the desired positioning. In such contexts, it may be desirable to restrain movement of the ablation catheter to ensure a desired orientation of the distal end of the catheter relative to the distal end of the sheath. For example, in rotating the distal end of the sheath to position the distal end of the catheter, it can be desirable for a maximum longitudinal length of the exposed distal end of the catheter to be fixed to improve control of the ablation catheter. Alternatively or additionally, it may be desirable to prevent the ablation catheter from rotating relative to the sheath about a longitudinal axis common to both the catheter and sheath to improve control of the ablation catheter. Arrangements such as just discussed may also or alternatively prevent the ablation catheter from retracting into the sheath or extending out of the sheath further than desired during a procedure.

Disclosed herein are various embodiments of devices, systems, and methods that can resolve, improve, or address one or more of the issues discussed above. In various embodiments, a retaining device is used to fix an elongated medical implement relative to a medical device that includes a sheath through which the elongated medical implement extends (or relative to at least a portion of the medical device, such as a hub thereof). In some instances, the retaining device may be provided as a retrofitting device that can be coupled to existing medical instruments to improve their functionality. While much of the following discussion is directed to embodiments of medical devices that can be used in cardiac electrophysiology procedures, it should be understood that the disclosure applies to other contexts, including vascular access procedures and instruments such as those discussed above. Advantages such as those previously mentioned and/or other advantages of the various embodiments will be evident from the disclosure herein.

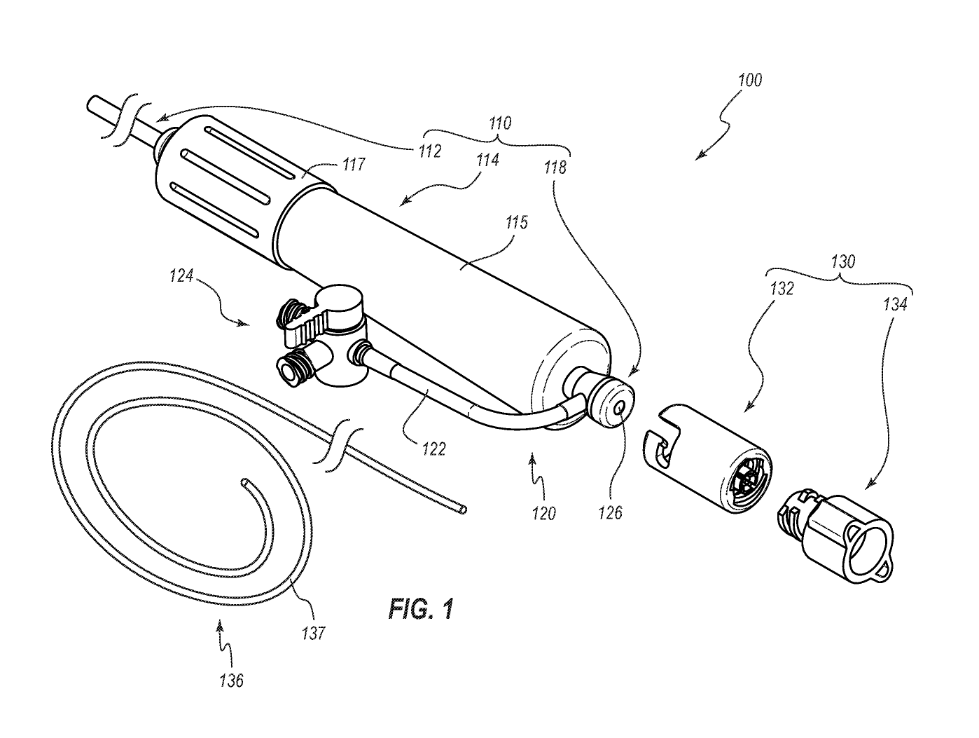

FIG. 1 is an exploded perspective view of an embodiment of a medical system 100 that includes an embodiment of a medical instrument 110, a retaining device 130, and an elongated medical implement 136 that can be inserted through the medical instrument 110 and the retaining device 130. In the illustrated embodiment, the medical instrument 110 and the elongated medical implement 136 can be used in electrophysiology procedures.

In particular, in the illustrated embodiment, the medical instrument 110 can be a steerable sheath instrument, which may also be referred to as a steerable introducer or as a guiding introducer. For example, the medical instrument 110 can comprise a steerable sheath marketed by St. Jude Medical of Saint Paul, Minn., or a steerable sheath of any other suitable manufacture. In particular, various embodiments of the medical instrument 110 may be configured for used with one or more of the Agilis™ introducers available from St. Jude Medical, including the Agilis™ NxT, which are presently available in various curl sizes, including small (16.8 mm), medium (22.4 mm), and large (50.0 mm); the Agilis™ NxT II; or the Agilis™ EPI.

The illustrated medical instrument 110 includes a sheath 112, a body 114, and a hub 118. The sheath 112 can be a steerable sheath, such that a distal portion thereof can be selectively reconfigured via the proximally-positioned body 110. In some embodiments, the distal end of the sheath 112 can be curled or deflected within a plane that passes through a longitudinal axis defined by the sheath. In particular, in some embodiments, the sheath 112 can be curled in a first direction (e.g., a clockwise direction) within the plane, and may be configured to sweep through an angle greater than or equal to about 180 degrees. In other or further embodiments, the sheath 112 can be curled or deflected in a second direction (e.g., a counterclockwise direction). In various embodiments, the sheath 112 can be curled in the second direction through an angle greater than or equal to 90 degrees. Other deflection angles and directions (e.g., multi-planar deflections) are contemplated.

The body 114 can include a handle 115 and a rotatable collar 117. In some embodiments, the body 114 can be gripped with the hand of a practitioner, and the distal end of the sheath 112 can be deflected in one or more directions, as just discussed, by rotating the collar 117 in a clockwise or counterclockwise direction relative to the body 114.

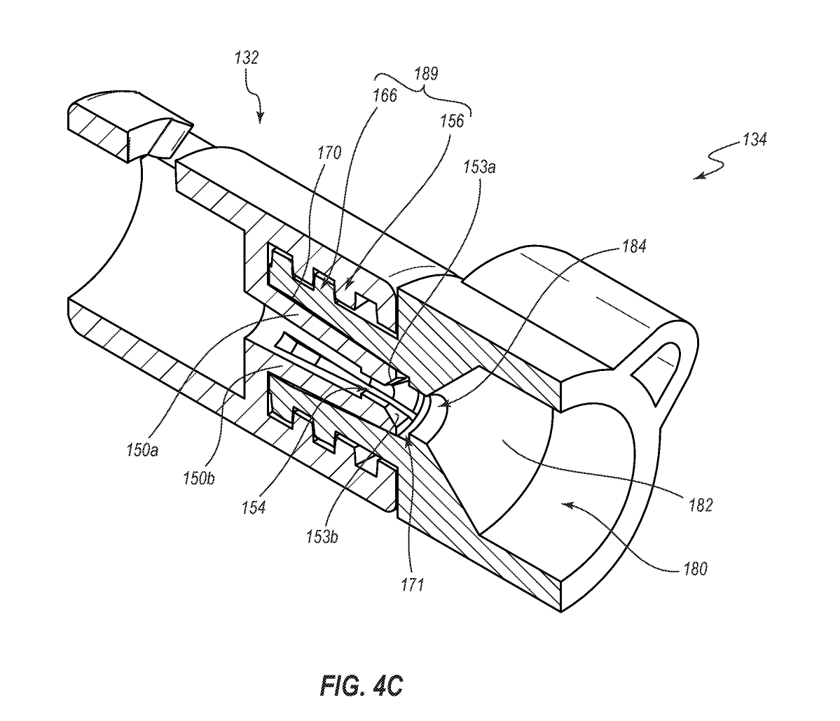

In the illustrated embodiment, the hub 118 extends from a proximal end of the body 114. The illustrated hub 118 is fitted with a hemostasis valve 126 to reduce or prevent blood loss during catheter introduction and/or exchange. The hub 118 includes a sideport 120. In the illustrated embodiment, the sideport 120 includes a flexible tube 122 that is coupled at one end thereof with a protrusion 190 (see FIG. 5A) and is coupled at an opposite end thereof with a plurality of ports. In particular, a proximal end of the flexible tube 122 is coupled with a three-way stopcock 124, which can be used for aspiration and/or fluid infusion, as is known in the art.

In some embodiments, the medical instrument 110 can include distal vent holes (not shown) to facilitate aspiration and minimize cavitation. In other or further embodiments, the medical instrument 110 can include a radiopaque tip marker to improve fluoroscopic visualization.

The elongated medical implement 136 can be any suitable device, such as a guidewire, catheter, etc. In the illustrated embodiment, the elongated medical implement 136 is a catheter 137, such as an ablation catheter. However, in many other embodiments, the elongated medical implement 136 is a catheter suitable for use in any medical procedure in which a catheter is introduced into a patient via a sheath.

In use, the sheath 112 of the medical instrument 110 can be inserted into a patient in any suitable manner. By way of illustration, several examples of medical procedures will be discussed hereafter. It should be understood that this discussion is not intended to limit the application of the restraining device 130 or systems in which it is employed.

In some instances, the sheath 112 is inserted into an interior of or at an exterior of an organ, such as, for example, the heart of a patient. For example, the sheath 112 can be introduced into the atria, ventricles, aortic root, pericardial space, etc., in known manners. A catheter 137, such as an ablation catheter, can be advanced through the sheath 112 to a desired region within the patient, and the sheath 112 can be used to steer a distal end of the catheter 137, as desired.

In some illustrative medical procedures, a guidewire is initially inserted into a desired region of the patient. A dilator (not shown) may be inserted through the hub 118, the body 114, and the sheath 112 until a distal end of the dilator, which can have an atraumatic tip to reduce the potential for trauma during punctures, extends from the sheath 112. The dilator and sheath 112 can be inserted into the patient over the guidewire, and in some instances, fluoroscopy may be used to ensure that the sheath 112 is present in the desired region.

With the sheath 112 in a desired position, the catheter 137 can be inserted through the hub 118, the body 114, and the sheath 112 into the desired space. A distal end of the catheter 137 (which, in some illustrative procedures, may be configured for ablation), can be manipulated to a desired initial position by one or more of advancing the distal end of the catheter 137 past the distal end of the sheath 112 and/or retracting a portion of the catheter 137 into the sheath 112 by a desired amount, rotating the collar 117 of the body 114 relative to the handle 114 in a desired direction and by a desired amount to achieve a desired deflection of the distal end of the sheath 112, or rotating the handle 115 about a longitudinal axis thereof to effect an associated rotation of the sheath 112 and an accompanying movement of the distal end of the catheter 137. One or more of the same movements may be used during the course of an operative procedure, such as an ablation. In some instances, the movements of the distal end of the catheter 137 are monitored via fluoroscopy.

With continued reference to FIG. 1, in the absence of the retaining device 130, it may be difficult to achieve or retain the distal end of the catheter 137 in a desired position or configuration. For example, in some instances, a practitioner may be preoccupied with holding both a distal portion of the catheter 137 and the handle 114 to maintain a desired relative orientation between these items and thus to ensure a desired position of the distal end of the catheter 137 within the patient. In some instances, merely holding the handle may permit the catheter 137 to move relative to the medical instrument 110 in undesired fashions, such as by, for example, inadvertently retracting into or advancing out of the sheath 112. Moreover, in some instances, a practitioner may use both hands to hold the handle 115 and manipulate the collar 117, and may find it impossible or impractical to simultaneously hold the proximal end of the catheter 137 while adjusting the deflection of the sheath 112 via the collar 117. In some instances, the resultant movement of the sheath 112 may result in inadvertent movements of the catheter 137, such as, for example, inadvertent retraction into or advancement out of the sheath 112.

In various instances, the retaining device 130 can ameliorate or resolve one or more of the foregoing issues. These and/or other advantages of the retaining device 130 will be evident from the present disclosure. As discussed more fully hereafter, the retaining device 130 can be configured to lock the catheter 137 relative to the hub 118. Thus, in some instances, the retaining device 130 can prevent the catheter 137 from being advanced beyond the distal end of the sheath 112 and/or from being retracted into the sheath 112 in undesirable manners. The retaining device 130 can prevent the catheter 137 from rotating relative to the medical instrument 110 about a longitudinal axis defined thereby.

In the illustrated embodiment, the retaining device 130 includes an adapter 132 and a locking member 134. As further discussed below, the adapter 132 can be configured to couple with the hub 118 of the medical instrument 110. In various embodiments, the locking member 134 can cooperate with the adapter 132 to selectively lock the catheter 137 relative to the medical instrument 110 or selectively release the catheter 137 to move freely relative to the medical instrument 110.

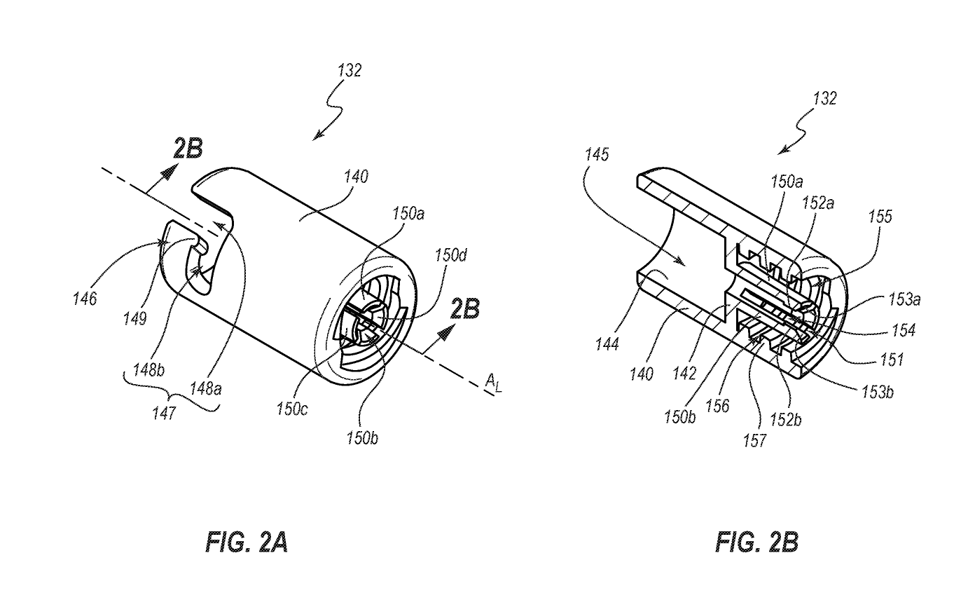

FIGS. 2A and 2B depict the illustrated adapter 132 in greater detail. The adapter 132 includes a sidewall 140 and a partition 142. In the illustrated embodiment, the sidewall is shaped substantially as a hollow cylinder of uniform diameter. The illustrated partition 142 is a substantially planar element that substantially bisects the sidewall 140 and has a thickness approximately equal to that of the sidewall 140. Any other suitable arrangements of the sidewall 140 and or the partition 142 are contemplated.

A distal portion of the adapter 132 is configured to couple with the medical instrument 110, and a proximal portion of the adapter 132 is configured to couple with the locking member 134. The coupling arrangements for attaching the adapter 132 to the hub 118 and for interfacing with the locking member 134 depicted in FIGS. 1-7 are merely illustrative. That is, although inventive concepts are present in the specific arrangements illustrated in these drawings and those discussed with respect thereto, the couplings may likewise be achieved in other suitable manners.

In the illustrated embodiment, the distal portion of the adapter 132 defines a cavity 145 that is configured to receive at least a portion of the hub 118 of the medical instrument 110 therein, as discussed further below. In particular the cavity 145 is defined by an inner surface 144 of the adapter 132. The inner surface 144 includes an inner surface of the distal portion of the sidewall 140 and a distal surface of the partition 142. In some embodiments, at least a portion of the inner surface 144 is substantially complementary to an outer surface of the hub 118 so as to readily receive the hub 118. For example, in the illustrated embodiment, the portion of the inner surface 144 defined by the sidewall 140 is substantially cylindrical, and an outer surface of the hub 118 is likewise substantially cylindrical. Other arrangements of the hub 118 and the inner surface 144, whether complementary or otherwise, are contemplated.

In some embodiments, the inner surface 144 is dimensioned approximately the same as the outer surface of the hub 118 or is otherwise configured for a friction fit engagement with the hub 118. For example, in some embodiments, an inner diameter of the surface 144, which is substantially cylindrical in the illustrated embodiment, is approximately the same as an outer diameter of a substantially cylindrical portion of the hub 118. In other embodiments, the inner surface 144 is dimensioned larger (e.g., slightly larger) than the outer diameter of the hub 118. Such arrangements may facilitate rotation of the adapter 132 relative to the hub 118 when the hub 118 has been received within the cavity 145.

The adapter 132 can include an attachment mechanism 146 that is configured to secure the adapter 132 to the medical instrument 110. In the illustrated embodiment, the attachment mechanism 146 is configured to engage the sideport 120 of the hub 118. The attachment mechanism 146 can include a slot 147 into which at least a portion of the sideport 120 is received. The slot 147 can be defined by the sidewall 140.

In the illustrated embodiment, a first branch, or first portion 148a, of the slot 147 extends in a longitudinal direction and a second branch, or second portion 148b, of the slot 147 extends laterally from a proximal end of the first portion 148a. The first portion 148a includes an opening at a distal end of the sidewall 140 at which the sideport 120 can initially be received into the slot. As discussed further below, the sideport 120 can be advanced in a longitudinal direction to a proximal end of the first portion 148a of the slot 147, at which point the adapter 132 can be rotated relative to the hub 118 to advance the sideport 120 into the second portion 148b of the slot 147.

In some embodiments, the attachment mechanism 146 includes a stop that is configured to retain the sideport 120 within the slot 147. In the illustrated embodiment, the stop comprises a protrusion 149 that reduces a size of the second portion 148a of the slot relative to an adjacent, end portion thereof in which the sideport 120 is configured to be retained. The protrusion 149 may be said to constrict a region of the slot 147.

In the illustrated embodiment, the portion of the sidewall 140 that defines the cavity 145 is substantially continuous and, other than at the region defining the slot 147, fully encircles the portion of the hub 118 that is received therein. In other embodiments, the sidewall 140 may be discontinuous, such that only one, two, three, or some other number of sidewall panes extend distally (e.g., distally from the partition) to define a boundary of the cavity 145. Other suitable arrangements are also possible.

With continued reference to FIGS. 2A and 2B, the proximal portion of the illustrated adapter 132 includes a plurality of engagement members 150a, 150b, 150c, 150d that are configured to transition between an open or unlocked state and a closed or locked state to selectively engage the elongated medical implement 136. In particular, the illustrated embodiment includes four engagement members. Other numbers of engagement members are also possible (e.g., two, three, five, six). The engagement members 150a, 150b, 150c, 150d can define a lumen 154 through which the elongated medical implement 136 can extend.

In the illustrated embodiment, each engagement member 150a, 150b, 150c, 150d is an elongated stem having a distal end that is joined to the partition 142 and a proximal end that is capable of being deflected radially inwardly. The engagement members 150a, 150b, 150c, 150d are shown in the open state in FIGS. 2A and 2B. In this embodiment, the engagement members 150a, 150b, 150c, 150d extend longitudinally from the partition 142 and are substantially parallel to a longitudinal axis A_{L} defined by the adapter 132. As further discussed below, the adapter 132 can cooperate with the locking member 134 to transition the engagement members 150a, 150b, 150c, 150d from the open state to the locked state. When the engagement members 150a, 150b, 150c, 150d are transitioned to the locked state, the proximal ends of the engagement members can move radially inwardly toward the longitudinal axis A_{L}.

A separate slit 151 can be defined between each pair of adjacent engagement members 150a, 150b, 150c, 150d to facilitate movement of the engagement members relative to each other. In the illustrated embodiment, the lumen 154 defined by the engagement members 150a, 150b, 150c, 150d can have a minimum diameter when in the open state, and this minimum diameter can be reduced as the engagement members are moved to the locked state. In some instances, the proximal end of each engagement member 150a, 150b, 150c, 150d can come into contact with the proximal ends of adjacent engagement members 150c, 150d; 150c, 150d; 150a, 150b; 150a, 150b, respectively, when the engagement members 150a, 150b, 150c, 150d are transitioned to the locked state. In some instances, a size of the slits 151 can affect the size of the minimum diameter of the lumen 154 that can be achieved as the engagement members are transitioned to the locked state. Stated otherwise, the size of the slits 151 can affect the cross-sectional size of the smallest elongated medical implement 136 that can be engaged by the engagement members 150a, 150b, 150c, 150d. In some instances, such as when relatively larger (wider) elongated medical implements 136 are used, the locked state may be achieved without the proximal ends of the engagement members 150a, 150b, 150c, 150d coming into contact with each other.

In the illustrated embodiment, each engagement member 150a, 150b, 150c, 150d includes a projection, barb, or tooth 152a, 152b that extends radially inwardly. The teeth 152a, 152b can assist in gripping the elongated medical implement 136 when the engagement members 150a, 150b, 150c, 150d are transitioned to the closed state. In some embodiments, the engaging surfaces of the teeth can have one or more gripping features, such as grooves, coatings, and/or a film liner to enhance the friction that arises between the teeth and the elongated medical implement 136. In some embodiments, the proximal ends of the engagement members 150a, 150b, 150c, 150d can be angled or chamfered to define an entrance surface 153a, 153b that assists in introducing the elongated medical implement 136 into the lumen 154.

The engagement members 150a, 150b, 150c, 150d can be resiliently deformable to permit repeated transitioning between the open and locked states. In some embodiments, the adapter 132 is formed of a single, monolithic piece of material. In some embodiments, the material can be sufficiently rigid to permit secure attachment of the adapter 132 to the medical instrument 110, when the material is in a configuration such as depicted with respect to the distal end of the adapter 132, and the material can be sufficiently flexible and resilient to permit the engagement members to be resiliently deformed, when in a configuration such as that depicted with respect to the engagement members 150a, 150b, 150c, 150d. In various embodiments, the adapter 132 can be formed of one or more of acrylonitrile butadiene styrene (ABS), nylon, polypropylene, polycarbonate, polyvinyl chloride (PVC), polyethylene, acrylic, stainless steel, nickel plated brass, and/or one or more elastomers, such as silicone, ethylene polypropylene diene monomer (EPDM) rubber, or polyurethane.

With continued reference to FIGS. 2A and 2B, a proximal end of the adapter 132 can define a recess or cavity 155 into which a portion of the locking member 134 can be received. In the illustrated embodiment, the cavity is defined by an inner surface of the sidewall 140, a proximal surface of the partition 142, and an outer surface of the engagement members 150a, 150b, 150c, 150d. The adapter 132 can include a coupling mechanism 156 via which the adapter 132 can be secured to the locking member 134. In the illustrated embodiment, the coupling mechanism 156 includes a length of threading 157 that extends about an inner surface of the sidewall 140.

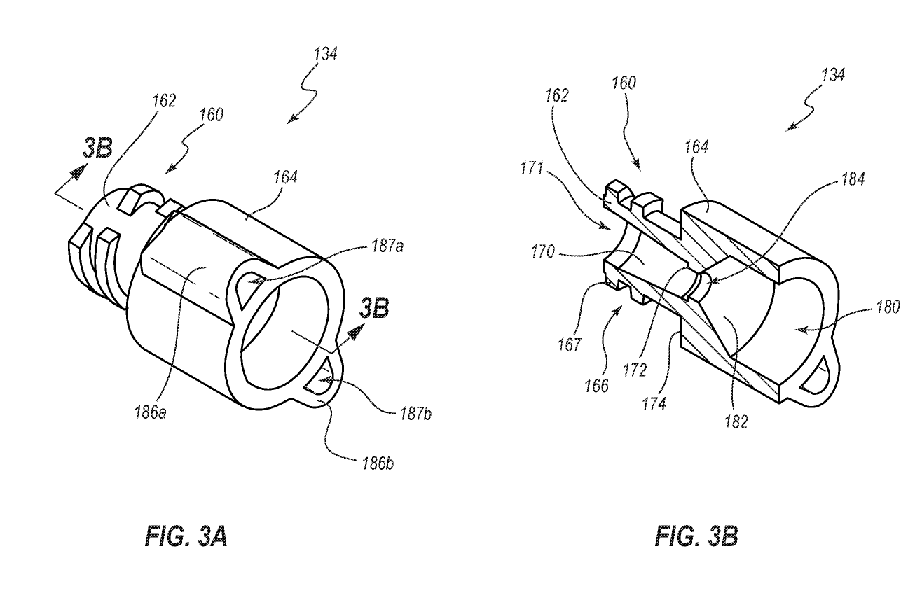

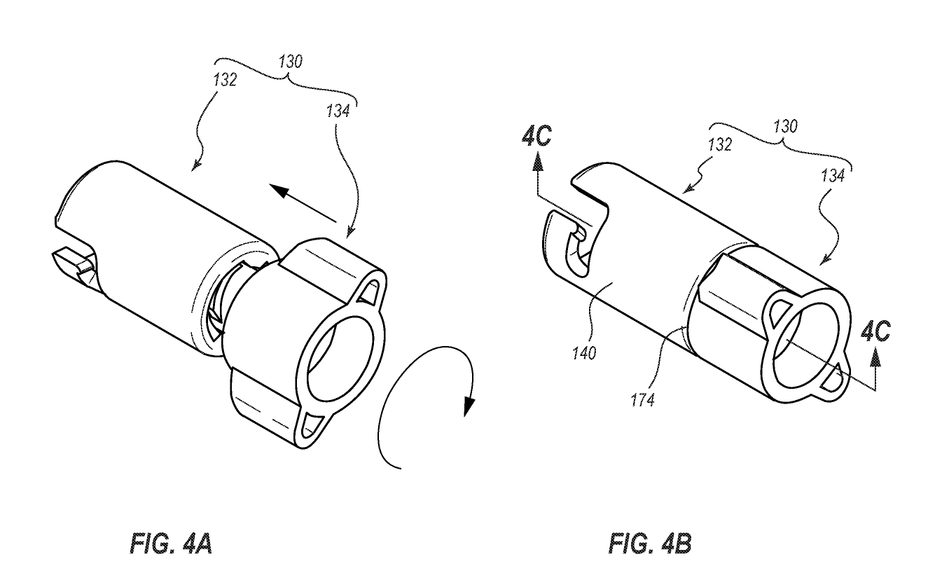

With reference to FIGS. 3A and 3B, the locking member 134, which may also be referred to as a locking nut, includes a distal portion that is configured to engage the proximal portion of the adapter 132. In the illustrated embodiment, the locking member 134 includes a distal projection 160 that is sized and otherwise configured to be received within the cavity 155 of the adapter 132. The projection 160 includes a sidewall 162 that extends distally from a broader sidewall 164 at a proximal end of the adapter 132. A coupling mechanism 166 is positioned at an exterior of the sidewall 162. In the illustrated embodiment, the coupling mechanism 166 includes a length of threading 167 that is complementary to the threading 157 of the adapter 132. In other embodiments, one of the coupling mechanisms 156 (FIG. 2B), 166 (FIG. 3B) may instead comprise a lug or other suitable arrangement that is configured to interact with the threading portion of the other coupling mechanism 156, 166. Other suitable coupling mechanisms are also possible. As shown in FIG. 4C, the coupling mechanisms 156, 166 may form a coupling interface 189 via which the adapter 132 and the locking member 134 are joined together. Any suitable coupling interface 189 is contemplated. As further discussed below, the coupling interface 189 can permit the adapter 132 and the locking member 134 to rotate relative to each other. In certain embodiments, rotation of the locking member 134 in a first direction relative to the adapter 132 can cause the locking member 134 to translate into closer proximity to the adapter 132. In further embodiments, rotation of the locking member in a second direction relative to the adapter 132 can cause the locking member 134 to translate away from the adapter 132.

The sidewall 162 can include an inner surface 170 that defines a cavity 171 into which the engagement members 150a, 150b, 150c, 150d are received when the locking member 134 is coupled to the adapter 134. The inner surface 170 may also be referred to as a camming surface, a biasing surface, or a ferrule surface. In the illustrated embodiment, the biasing surface 170 is shaped substantially as a lateral surface of a conical frustum. A diameter of the biasing surface 170 decreases in the proximal direction. Accordingly, as the engagement members 150a, 150b, 150c, 150d are advanced deeper into the cavity 171, or stated otherwise, are advanced further along the biasing surface 170, the proximal ends of the engagement members 150a, 150b, 150c, 150d are compressed toward the longitudinal axis of the adapter 132 and are transitioned to the locking state.

In the illustrated embodiment, a ledge 172 is present at a proximal end of the cavity 171. In some instances, the ledge 172 can act as a stop against which the engagement members 150a, 150b, 150c, 150d can rest when fully inserted into the cavity 170. In other or further embodiments, a ledge 174 defined by an external surface at a distal end of the sidewall 174 can interact with a proximal end of the sidewall 140 of the adapter 132 to control a maximum distance to which the engagement members 150a, 150b, 150c, 150d can be advanced within the cavity 170.

An inner surface of the sidewall 164 can define an insertion cavity 180 through which the elongated medical implement 136 can be introduced into the locking member 134, or more generally, introduced into the retaining device 130. At a distal end of the cavity 180, the inner surface of the sidewall 164 can include a guiding surface 182 that is sloped inwardly at an angle relative toward an opening 184 through which the elongated medical implement 136 is inserted. The guiding surface 182 and the opening 184 can align the elongated medical implement 136 with the lumen 154 defined by the engagement members 150a, 150b, 150c, 150d. (See also FIG. 4C.)

In certain embodiments, the locking member 134 can include gripping features that aid in tightening or loosening the locking member 134 relative to the adapter 132. In the illustrated embodiment, the locking member 134 includes a pair of grips 186a, 186b that extend radially outward from the sidewall 164. The illustrated grips 186a, 186b are at opposite sides of the sidewall 164. Other numbers and arrangements of grips are also possible. In other or further embodiments, the adapter 132 can include one or more grips.

In the illustrated embodiment, each grip 186a, 186b is hollow with an opening 187a, 187b extending longitudinally through the grip. In some instances, such an arrangement can reduce the material costs associated with producing the adapter 132.

As with the adapter 132, in some embodiments, the locking member 134 is formed of a single, monolithic piece of material. In various embodiments, the locking member 134 can be formed of one or more of acrylonitrile butadiene styrene (ABS), nylon, polypropylene, polycarbonate, polyvinyl chloride (PVC), polyethylene, acrylic, stainless steel, nickel plated brass, and/or one or more elastomers, such as silicone, ethylene polypropylene diene monomer (EPDM) rubber, or polyurethane. In some embodiments, the adapter 132 is made of PVC and the locking member 134 is made from polycarbonate. In certain embodiments, it may be advantageous to use dissimilar materials in threaded fittings due to galling and/or stiction that might otherwise occur between sliding surfaces.

FIG. 4A depicts the adapter 132 and the locking nut 134 of the retaining device 130 engaged with one another and in an open state in which the elongated medical implement 136 can be readily advanced through the retaining device 130. As depicted by the arrows in this drawing, rotation of the locking member 134 in the clockwise direction relative to the adapter 132 can translate the locking member 134 toward the distal end of the adapter 132. Further rotation can advance the retaining device 130 to a locked state in which the elongated medical implement 136 is fixed within the retaining device 130.

FIGS. 4B and 4C depict the retaining device in the locked state. In the illustrated embodiment, the ridge 174 of the locking member 134 is brought into contact with the distal end of the sidewall 140 of the adapter 132 when the locking state is achieved, as shown in FIG. 4B. FIG. 4C illustrates the coupling interface 189. In particular, the coupling mechanism 166 of the locking member 134 is shown as having interacted with the coupling mechanism 156 of the adapter 132 to draw the engagement members 150a, 150b into the cavity 171. In moving to the illustrated position, the proximal ends of the engagement members 150a, 150b were increasingly deflected by the biasing surface 170, thereby reducing the size of the lumen 154.

FIGS. 5A and 5B depict stages of coupling the retaining device 130 with the medical instrument 110. The retaining device 130 can be coupled with the medical instrument 110 at any suitable time. For example, in the illustrated coupling event, the retaining device 130 is coupled with the medical instrument 110 prior to inserting the elongated medical implement 136 into the medical instrument 110. In other instances, the retaining device 130 may be coupled with the medical instrument 110 after the elongated medical implement 136 has been inserted into the medical instrument 110, such as by advancing the retaining device 130 distally over the elongated medical implement 136 to the orientations shown in FIGS. 5A and 5B. In the illustrated coupling event, the adapter 132 and the locking member 134 are in the open state during coupling of the adapter 132 with the medical instrument 110.

As previously discussed, the hub 118 can include a sideport 120, which can include a flexible tube 122 coupled with a lateral boss or protrusion 190. In some embodiments, the flexible tube 122 is attached to an exterior of the protrusion 190, and the flexible tube 122 may be more compliant than the protrusion 190.

As shown in FIG. 5A, in an initial stage of coupling the adapter 132 to the medical instrument 110, the longitudinal axis A_{L} of the adapter 132 can be aligned with a longitudinal axis of the medical instrument 110. The adapter 132 can be advanced over at least a portion of the hub 118. In particular, a portion of the hub 118 can be received into the distal cavity 145 (FIG. 2B) of the adapter 132. Further, a portion of the sideport 120 can be advanced proximally through the first portion 148a of the slot 147.

As shown in FIG. 5B, with the sideport 120 extending through the proximal end of the first portion 148a of the slot 147, the adapter 132 can be rotated relative to medical instrument 110 to introduce the sideport 120 into the second portion 148b of the slot 147. In some instances, the locking protrusion 149 can compress the flexible tubing 120 as the sideport 120 is advanced past the protrusion 149 and/or advancement of the sideport 120 past the protrusion 149 can cause the portion of the adapter 132 that is positioned distally relative to the second portion 148b of the slot 147 to deflect in a distal direction to provide sufficient room in the slot 149 for the sideport 120 to be advanced to the end of the second portion 148b of the slot 147. Thereafter, the locking protrusion 149 can aid in retaining the sideport 120 within the second portion 148b of the slot 147. Such an arrangement can resist decoupling of the adapter 132 from the medical instrument 110 as the locking member 134 is rotated in either direction about the longitudinal axis A_{L} for tightening or loosening the locking member 134 relative to the adapter 132. In the illustrated arrangement, attaching the adapter 132 to the medical instrument 110 is achieved by rotating the adapter 132 in a first direction (e.g., clockwise) relative to the medical instrument 110, and tightening of the locking member 134 relative to the adapter 132 is achieved by rotating the adapter 132 in the same direction (e.g., clockwise) relative to the adapter 132. Such an arrangement can assist in preventing inadvertent decoupling of the adapter 132 from the medical instrument 110 during tightening of the locking member 134, as any excess rotational forces that may be imparted to the adapter 132 during such tightening (including any inadvertent over tightening) would merely tend to force an end portion of the slot 149 against the sideport 120, rather than advancing the sideport 120 in the opposite direction toward the first portion 148a of the slot 147.

In certain embodiments, the retaining device 130 can be selectively removed from the medical instrument 110. For example, in the illustrated embodiment, the adapter 132 can be removed from the medical instrument 110 by reversing the procedures discussed with respect to FIGS. 5A and 5B.

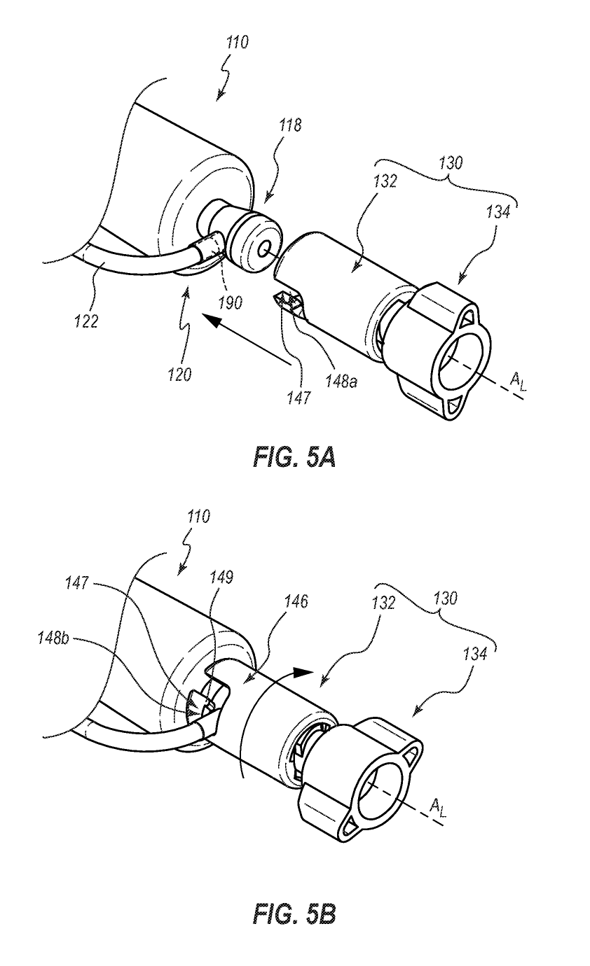

FIG. 6 illustrates the medical instrument 110 and retaining device 130 after the elongated medical implement 136 has been inserted through them and after the retaining device 130 has been transitioned to the closed state. When in the closed state, as illustrated, the retaining device 130 grips the elongated medical implement 136 to prevent longitudinal and/or rotational movement of the elongated medical implement 136 relative to the retaining device 130. Due to the fixed relationship between the retaining device 130 and the medical instrument 110, the retaining device 130 likewise generally prevents longitudinal and/or rotational movement of the elongated medical implement 136 relative to the medical instrument 110. For example, the retaining device 130 generally prevents longitudinal and/or rotational movement of the elongated medical implement 136 relative to the hub 118. However, as previously discussed, in some embodiments, the sheath 112 of the medical device 110 is capable of being curled or otherwise deflected at a distal end thereof. Although a proximal portion of the elongated medical implement 136 may be fixed, such distal movement of the sheath 112 can cause a limited amount of relative longitudinal movement between the distal end of the elongated medical implement 136 and the distal end of the sheath 112. Such relative movement may be minimal or negligible, in some instances. In other instances, such movement may be accounted for or even adjusted by the practitioner. For example, in some instances, the elongated medical implement 136 may be advanced beyond the distal end of the sheath 112 by a desired amount; the sheath 112 may be readjusted, which may alter the relative positions of the sheath 112 and the implement 136; and the practitioner may then open the retaining device 130, readjust the elongated medical implement 136 relative to the sheath 112 to achieve a desired orientation, and then relock the retaining device 130.

In certain embodiments, the elongated medical implement 136 includes a marking system 192 that provides information regarding a distance to which the elongated medical implement 136 extends beyond the distal end of the sheath 112. In the illustrated embodiment, the elongated medical implement 136 is a catheter 137 (e.g., ablation catheter) that includes a marking system 192 in the form of a series of graduations 194a, 194b positioned thereon. The graduations may be spaced from each other by regular intervals (e.g., millimeters and/or centimeters). The graduations 194a, 194b can represent length, and may include rulings (such as in millimeters, centimeters, and/or inches).

In certain embodiments, the marking system 192 accounts for or is otherwise dedicated to a particular medical instrument 110/retaining device 130 combination. Stated otherwise, in some embodiments, the marking system 192, as applied to an elongated marking system 192, is dedicated for use with a specific medical instrument 110/retaining device 130 combination, given that other medical instrument 110/retaining device 130 combinations may have a longer or shorter overall length when coupled with each other. For example, in some embodiments, a starting point or a zeroing of the marking system 192 (e.g., a first graduation 194 or a point from which all graduations 194 are measured on the elongated medical implement 136) may account for a combined length L_{A} of the medical device 110 and the retaining device 130 (e.g., the retaining device 130 in the closed state). Accordingly, a length L₁ to which the catheter 137 extends beyond the distal end of the sheath 112 can be determined from the marking system 192, such as by determining a length or number of graduations 194a that extend distally into the retaining device 130 or beyond, or stated otherwise, by determining the length of the marking system 192, such as the number of graduations 194a, that extend distally relative to a proximal end of the retaining device 130. The determination may also be made merely by reading off a value (e.g., a ruled marking, such as 2 millimeters, 3 millimeters, etc.) from a ruled marking system.

In other or further embodiments, the marking system 192 may be used for multiple medical instrument 110/retaining device 130 combinations that may have different combined lengths L_{A}. For example, in some embodiments, at least a proximal region of the elongated medical implement 136 can include the marking system 192, which may itself include a series of graduations 194. In some embodiments, each graduation 194 indicates a distance from the distal tip of the elongated medical implement 136. Thus, in some embodiments, by knowing a value of the length L_{A} of a combined medical instrument 110/retaining device 130 combination with which the elongated medical implement 136 is being used, a practitioner can determine what amount of the elongated medical implement 136 extends beyond the distal end of the sheath 112 by reading or otherwise obtaining the value of the appropriate graduation (e.g., the graduation at the proximal end of the retaining device 130) and subtracting from this value the length L_{A}.

In some embodiments, the marking system 192 can any suitable indicia to identify the specific lengths L_{A} of one or more known medical instrument 110/retaining device 130 combinations. For example, the marking system 192 can include one or more lines, arrows, or other suitable indicia with accompanying text, coloring, etc. to identify one or more lengths L_{A}, as measured from the distal end of the elongated medical implement 136. In further embodiments, the marking system 192 may include a plurality of separate sets of graduations 194, with each set of graduations beginning at a known length L_{A}—e.g., beginning at the indicia representing the length L_{A}—of a specific and different medical instrument 110/retaining device 130 combination.

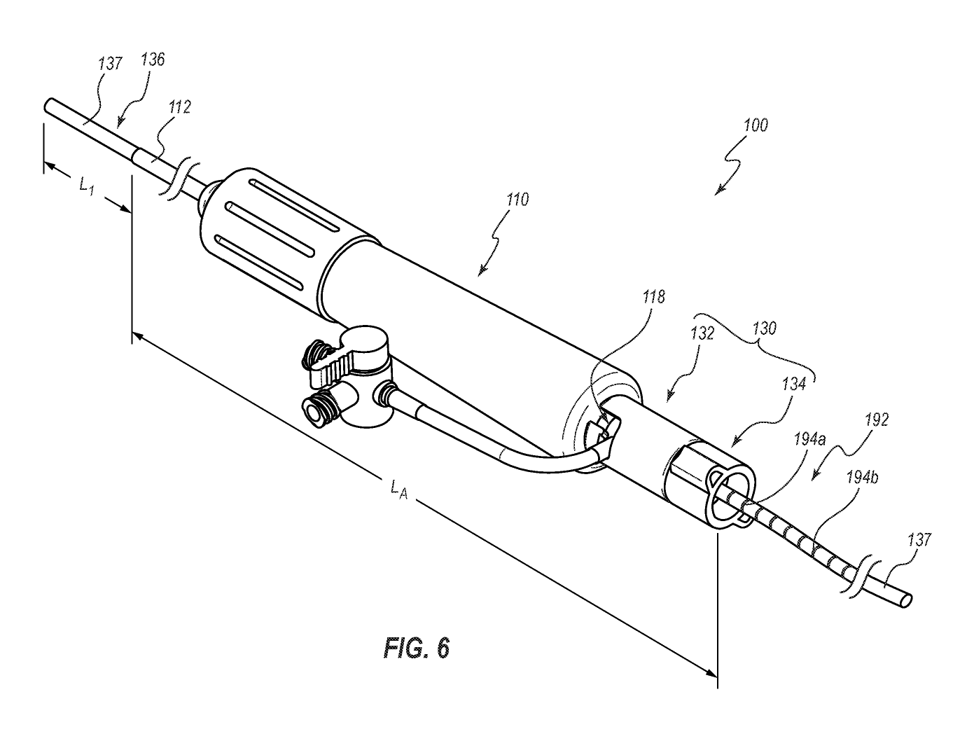

FIG. 7 depicts the same arrangement as that of FIG. 6, except that the retaining device 130 has been transitioned to the open state to permit distal movement of the catheter 137 and then returned to the locked state to prevent further movement of the catheter 137. As can be seen by comparing FIGS. 6 and 7, the graduation 194a has been moved distally to an interior of the retaining device 130. The new length L₂ can be determined from the graduation 194b, which is positioned at the extreme proximal end of the locking mechanism 134 (i.e., at the extreme locking end of the illustrated retaining device 130)

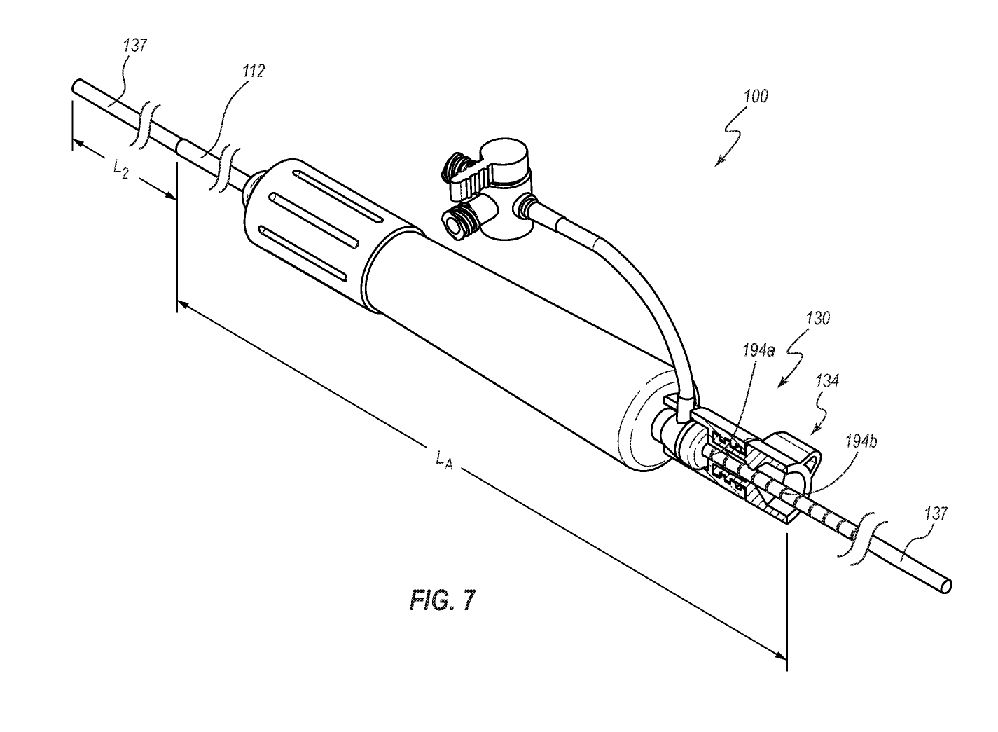

FIG. 8 depicts another embodiment of a retaining device 230 that can resemble the retaining device 130 described above in certain respects. Accordingly, like features are designated with like reference numerals, with the leading digits incremented to “2.” Relevant disclosure set forth above regarding similarly identified features thus may not be repeated hereafter. Moreover, specific features of the retaining device 230 may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the retaining device 230. Any suitable combination of the features and variations of the same described with respect to the retaining device 130 can be employed with the retaining device 230, and vice versa. This pattern of disclosure applies equally to further embodiments depicted in subsequent figures and described hereafter, wherein the leading digits may be further incremented.

The retaining device 230 can include an adapter 232 and a locking member 234. In the illustrated embodiment, the adapter 232 includes a proximal projection 261 that is similar to the distal projection 160 of the locking member 134. The proximal projection 261 defines a biasing surface 270 that resembles the biasing surface 170 discussed above.

The locking member 234 defines a cavity 255, such as the cavity 155 discussed above, into which the proximal projection 261 can be received. The locking member 234 includes a plurality of engagement members (three of the engagement members 250a, 250b, 250c are shown), such as the engagement members 150a, 150b, 150c, 150d discussed above.

The retaining device 230 functions in manners such as described above with respect to the retaining device 130. The primary difference between the retaining devices 130, 230 is the reversal of the biasing surface and the engagement members relative to the adapter and the locking member.

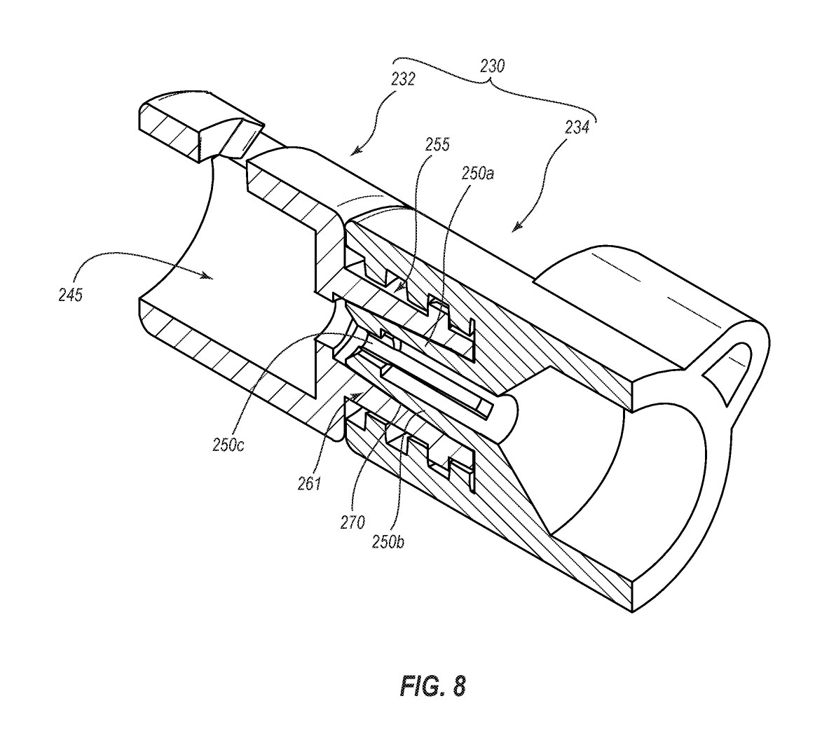

FIG. 9 is an elevation view of another embodiment of an adapter 332 that includes an attachment mechanism 346 that varies from the attachment mechanism 146 discussed above. The attachment mechanism 346 includes a slot 347 that includes three portions 348a, 348b, and 348c. The first and second portions 348a, 348b are analogous to the first and second portions 148a, 148b of the slot 147. The second portion 348b even includes a locking protrusion 349a, such as the locking protrusion 149. However, the third portion 348c permits the adapter 332 to be attached to the medical instrument 110 (FIGS. 5A and 5B) by rotating the adapter 332 in either direction (clockwise or counterclockwise). The third portion 348c includes a locking protrusion 349b that functions in the same manner as the locking protrusion 149 discussed above.

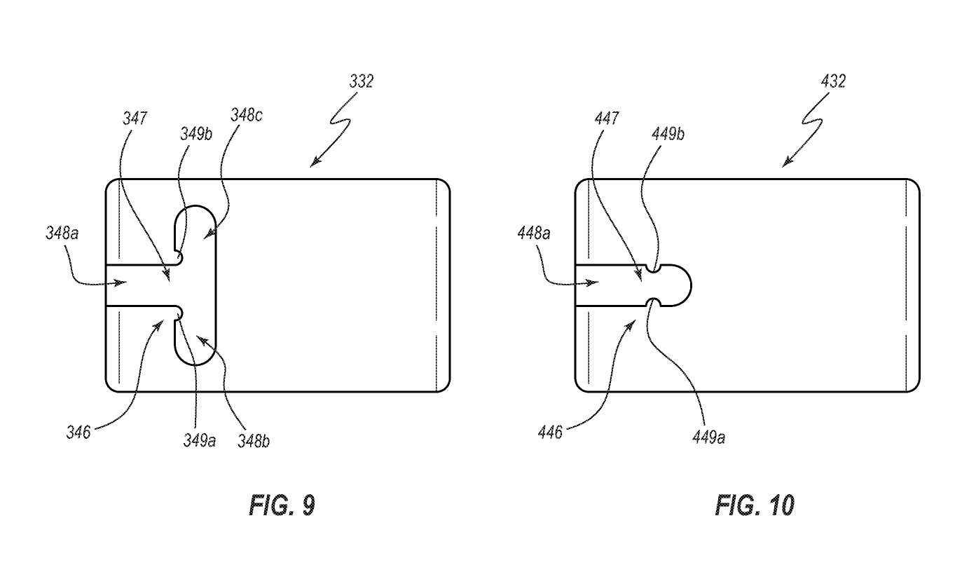

FIG. 10 is an elevation view of yet another embodiment of an adapter 432 that includes an attachment mechanism 446 that varies from the attachment mechanism 146 discussed above. The attachment mechanism 446 includes a slot 447 that includes a single portion 448a that extends longitudinally. The adapter 432 is secured to the sideport 120 of the medical instrument 110 (FIG. 1) merely by advancing the sideport 120 proximally past one or more locking protrusions 449a, 449b.

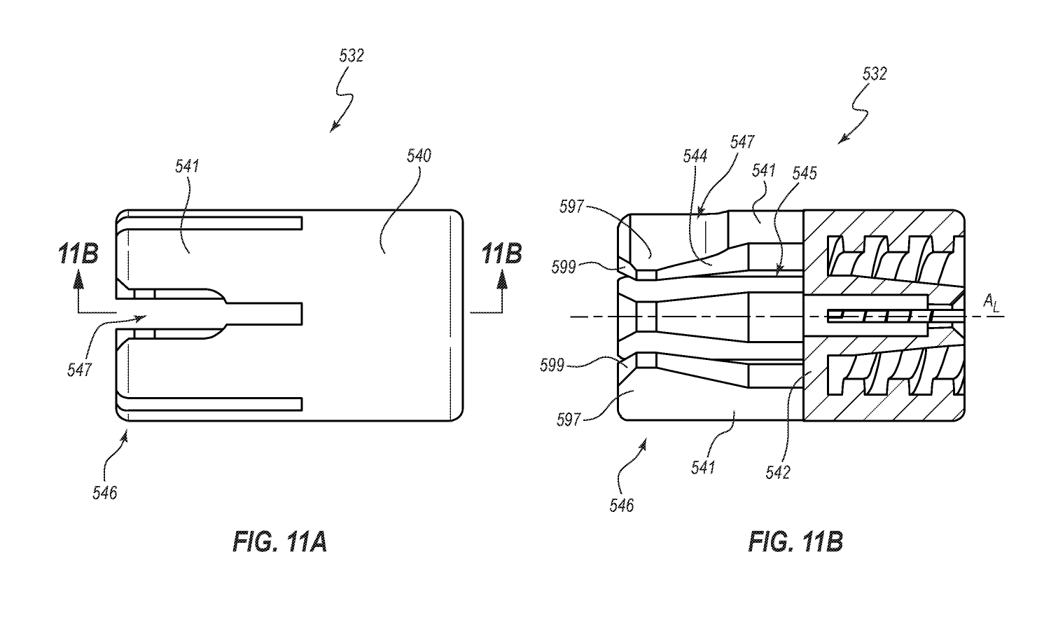

FIGS. 11A and 11B depict still another embodiment of an adapter 532. As with the other adapters disclosed herein, the adapter 532 is compatible with the medical system of FIG. 1, as well as other medical systems. The adapter 532 includes a sidewall 540 similar to the sidewall 140 discussed above, and also includes a partition 542 similar to the partition 142 discussed above. A proximal end of the adapter 532 is similar to the proximal end of the adapter 132, and is configured to be selectively attached to a locking member such as the locking member 134.

The adapter 532 includes an attachment mechanism 546 that varies from the attachment mechanism 146 discussed above. In particular, the attachment mechanism 546 includes a plurality of sidewall panels 541, two of which define a slot 547 into which a portion of the sideport 120 can be received. The panels 541 can extend distally from the partition 542 and can be resiliently flexible. The panels 541 can collectively cooperate with the partition 542 to define a cavity 545 into which a portion of the hub 118 (see FIGS. 1, 5A) can be received. In some embodiments, an inner surface 544 of the adapter 532, which can include inner surfaces of the partition 542 and the panels 541, is shaped substantially complementarily to an outer surface of the hub 118. For example, in the illustrated embodiment, the hub 118 is shaped substantially as a disk and a distally extending conical frustum; similarly, the inner surfaces 543 of the panels 541 are shaped as a cylindrical region for receiving a disk and as a distally extending conical frustum. In some embodiments, a snug fit may be achieved between the inner surface 544 and the hub 118. In other or further embodiments, the size of the cavity 545 when the panels 541 are in a natural, relaxed, or non-deformed state is slightly smaller than the portion of the hub 118 that is received therein, and the panels 541 may remain slightly deformed when the adapter 532 is attached to the hub 118. The slight deformation of the panels 541 may thus bias the panels 541 radially inward to securely grip the hub 118. Other arrangements are also possible.

In some embodiments, radially inwardly projecting stops or catches 597 may engage a distal collar of the hub 118. These portions of the panels 541 and/or an angled portion of the inner surface 544 that is positioned immediately adjacent to and proximal of the catches 597 can inhibit or prevent inadvertent detachment of the adapter 532 from the hub 118. Stated otherwise, one or more of these portions of the panels 541 can engage a distal surface of the hub 118 after the hub 118 has been received into the cavity 545 to resist detachment of the adapter 532 from the hub 118.

In some embodiments, the panels 541 include angled introducing surfaces 599 that assist in advancing the adapter 532 over the hub 118. In particular, the surface 599 may be angled relative to a longitudinal axis A_{L} of the adapter so as to deflect the panels 541 radially outwardly as the hub 118 is advanced into the cavity 545.

In some embodiments, the gripping engagement of the panels 541 with the hub 118 is sufficient to prevent rotation of the adapter 532 about the longitudinal axis A_{L}, such as when the locking member 134 is being advanced into or retracted from the proximal end of the adapter 532. In other or further embodiments, the slot 547 into which the sideport 120 of the hub 118 is received can abut the sideport 120 to prevent rotational movement of the adapter 532 relative to the hub 118.

As previously mentioned, while the drawings and written description have focused on illustrative devices, systems, and methods related to certain electrophysiology procedures, it is to be understood that embodiments may be used in any other suitable context. For example, although steerable sheaths are depicted in the drawings and discussed with respect thereto, in other embodiments, the sheaths may not be steerable. Moreover, the present disclosure is applicable in other contexts employing medical devices that have sheaths (whether steerable or non-steerable) and elongated medical implements, such as, for example, vascular access procedures. Moreover, it will be understood by those having skill in the art that changes may be made to the details of the above-described embodiments without departing from the underlying principles presented herein. For example, any suitable combination of various embodiments, or the features thereof, is contemplated.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

References to approximations are made throughout this specification, such as by use of the terms “about” or “approximately.” For each such reference, it is to be understood that, in some embodiments, the value, feature, or characteristic may be specified without approximation. For example, where qualifiers such as “about,” “substantially,” and “generally” are used, these terms include within their scope the qualified words in the absence of their qualifiers. For example, where the term “substantially planar” is recited with respect to a feature, it is understood that in further embodiments, the feature can have a precisely planar orientation.

Any reference throughout this specification to “certain embodiments” or the like means that a particular feature, structure or characteristic described in connection with that embodiment is included in at least one embodiment. Thus, the quoted phrases, or variations thereof, as recited throughout this specification are not necessarily all referring to the same embodiment or embodiments.

Similarly, it should be appreciated that in the above description of embodiments, various features are sometimes grouped together in a single embodiment, figure, or description thereof for the purpose of streamlining the disclosure. This method of disclosure, however, is not to be interpreted as reflecting an intention that any claim require more features than those expressly recited in that claim. Rather, as the following claims reflect, inventive aspects lie in a combination of fewer than all features of any single foregoing disclosed embodiment.

The claims following this written disclosure are hereby expressly incorporated into the present written disclosure, with each claim standing on its own as a separate embodiment. This disclosure includes all permutations of the independent claims with their dependent claims.

Recitation in the claims of the term “first” with respect to a feature or element does not necessarily imply the existence of a second or additional such feature or element. Elements specifically recited in means-plus-function format, if any, are intended to be construed in accordance with 35 U.S.C. §112(f). Embodiments of the invention in which an exclusive property or privilege is claimed are defined as follows.

1. A device for use with a medical instrument that comprises an elongated sheath and a hub that comprises a sideport, wherein the hub and the sheath are configured to permit at least a portion of an elongated medical implement to pass through them, the device comprising:
a plurality of engagement members that cooperate to define an opening through which the elongated medical implement can pass;
a biasing surface;
an adapter comprising one of (i) the plurality of engagement members or (ii) the biasing surface, the adapter further comprising:
an inner surface that defines a cavity for receiving at least a portion of the hub; and
an attachment mechanism configured to pass over the sideport and directly engage at least a portion of the hub to secure the adapter to the medical instrument; and
a locking member comprising the other of (i) the plurality of engagement members or (ii) the biasing surface, the locking member being configured to couple to the adapter and rotate relative to the adapter when coupled therewith such that rotation of the locking member in a first direction advances the plurality of engagement members along the biasing surface to transition the engagement members from an open state to a locked state,
wherein, when the elongated medical implement is positioned within the opening defined by the plurality of engagement members, the elongated medical implement is permitted to move relative to the hub when the plurality of engagement members are in the open state and is prevented from moving axially relative to the hub when the plurality of engagement members are in the locked state.

2. The device of claim 1, wherein the attachment mechanism of the adapter comprises a slot configured to receive at least a portion of the sideport of the hub therein.

3. The device of claim 1, wherein the attachment mechanism of the adapter permits the adapter to selectively attach to the medical instrument via the sideport of the hub and selectively detach from the medical instrument.

4. The device of claim 1, wherein the attachment mechanism of the adapter comprises a plurality of panels that are configured to resiliently deflect outwardly away from a longitudinal axis of the adapter as the hub is introduced into the adapter.

5. The device of claim 4, wherein the plurality of panels are configured to move inwardly toward the longitudinal axis of the adapter to secure the adapter to the hub.

6. The device of claim 1, further comprising threading via which the locking member is coupled to the adapter.

7. A system comprising:
a medical instrument that comprises an elongated sheath and a hub that comprises a sideport;
an elongated medical implement of which at least a portion is configured to pass through the sheath and the hub; and
a retaining device comprising:
a plurality of engagement members that cooperate to define an opening through which the elongated medical implement can pass;
a biasing surface;
an adapter comprising one of (i) the plurality of engagement members or (ii) the biasing surface, the adapter further comprising:
an inner surface that defines a cavity for receiving at least a portion of the hub; and
an attachment mechanism configured to pass over the sideport and directly engage at least a portion of the hub to secure the adapter to the medical instrument; and
a locking member comprising the other of (i) the plurality of engagement members or (ii) the biasing surface, the locking member being configured to couple with the adapter and rotate relative to the adapter when coupled therewith such that rotation of the locking member in a first direction advances the plurality of engagement members along the biasing surface to transition the engagement members from an open state to a locked state,
wherein, when the elongated medical implement is positioned within the opening defined by the plurality of engagement members, the elongated medical implement is permitted to move relative to the hub when the plurality of engagement members are in the open state and is prevented from moving axially relative to the hub when the plurality of engagement members are in the locked state.

8. The system of claim 7, wherein the attachment mechanism of the adapter comprises a slot into which at least a portion of the sideport of the hub is received.

9. The system of claim 7, wherein the attachment mechanism of the adapter permits the adapter to selectively attach to the medical instrument via the sideport of the hub and selectively detach from the medical instrument.

10. The system of claim 7, wherein the attachment mechanism of the adapter comprises a plurality of panels that are configured to resiliently deflect outwardly away from a longitudinal axis of the adapter as the hub is introduced into the adapter.

11. The system of claim 10, wherein the plurality of panels are configured to move inwardly toward the longitudinal axis of the adapter to secure the adapter to the hub.

12. The system of claim 7, wherein the retaining device further comprises threading via which the locking member is coupled to the adapter.

13. The system of claim 7, wherein rotation of the locking member in a second direction opposite the first direction transitions the engagement members from the locked state to the open state.

14. The system of claim 7, wherein, when the elongated medical implement is positioned within the opening defined by the plurality of engagement members, the elongated medical implement is prevented from rotating about a longitudinal axis defined by the elongated medical implement.

15. The system of claim 7, wherein the hub comprises a hemostasis valve.

16. The system of claim 7, wherein the elongated medical implement comprises a catheter.

17. A device for use with a medical instrument that comprises an elongated sheath and a hub that comprises a sideport, wherein the hub and the sheath are configured to permit at least a portion of an elongated medical implement to pass through them, the device comprising:
an adapter comprising a sidewall that defines at least a portion of a cavity configured to receive at least a portion of the hub, the sidewall comprising a slot into which at least a portion of the sideport of the hub is configured to be retained to secure the adapter to the medical instrument; and
a locking member configured to couple with the adapter and rotate relative to the adapter when coupled therewith such that rotation of the locking member in a first direction transitions the device from an open state to a locked state,
wherein, when the elongated medical implement is positioned within the device, the elongated medical implement is permitted to move relative to the hub when the device is in the open state and is prevented from moving axially relative to the hub when the device is in the locked state.

18. The device of claim 17, wherein the adapter comprises one of (i) a plurality of engagement members or (ii) a biasing surface and the locking member comprises the other of (i) the plurality of engagement members or (ii) the biasing surface, and wherein rotation of the locking member in the first direction advances the plurality of engagement members along the biasing surface to deflect a portion of the engagement members radially inward.
